(19) United States
(12) Patent Application Publication
(10) Pub. No.: US 2020/0330147 A1
(43) Pub. Date: Oct. 22, 2020

(54) ELECTROSURGICAL PENCIL WITH A PROTECTIVE GUARD

(71) Applicant: Covidien LP, Mansfield, MA (US)

(72) Inventors: Jacob C. Baril, Norwalk, CT (US); George S. Matta, Plainville, MA (US); Saumya Banerjee, Hamden, CT (US); Matthew A. Dinino, Newington, CT (US)

(21) Appl. No.: 16/850,454

(22) Filed: Apr. 16, 2020

Related U.S. Application Data
(60) Provisional application No. 62/835,601, filed on Apr. 18, 2019.

(51) Int. Cl. A61B 18/14; A61B 18/12

(52) U.S. Cl. CPC A61B 18/14; A61B 2018/00589; A61B 18/1206

(57) ABSTRACT

An electrosurgical pencil includes a body having proximal and distal ends. A treatment blade extends from the distal end of the body and electrically connects to an energy source. A protective guard operably engages the distal end of the body via a pivot pin and includes a pair of spaced-apart arms configured to receive the treatment blade. The protective guard is pivotable about the pivot pin between a closed position and a position for allowing treatment of tissue. A return electrode operably connects to the protective guard and to the electrosurgical energy source. A biasing member operably couples to the protective guard and is configured to bias the protective guard in the closed position.

### CROSS-REFERENCE TO RELATED APPLICATIONS

This application claims the benefit of U.S. Application Ser. No. 62/835,601, filed on Apr. 18, 2019, the entire disclosure of which is incorporated by reference herein.

### BACKGROUND

### Technical Field

The present disclosure relates to an electrosurgical instrument, and more particularly, to an electrosurgical pencil with a protective guard.

The coagulation of blood vessels is a necessary part of medical surgery and can be performed by an electrosurgical instrument commonly known as an electrosurgical pencil or coagulator pencil. With this type of pencil, an electrically conductive metal tip (e.g., in the form of a blade or a needle) extends outwardly from the distal end of the body of the pencil, the latter acting as a hand grip for a surgeon using the pencil. In use, the tissue of a patient is typically electrically connected to one side of an electrosurgical circuit via a return pad, and the electrically conductive tip is typically connected to the other side of the same circuit. When the metal tip touches or is near the tissue at the surgical site, a high frequency electrical current flows from the electrode to the tissue, thus coagulating and cauterizing the tissue.

Typical electrosurgical pencils allow the surgeon to change between two pre-configured settings (e.g., coagulation and cutting) via two discrete buttons disposed on the electrosurgical pencil itself. Other electrosurgical pencils allow the surgeon to increment the power applied when the coagulating or cutting activation button of the instrument is actuated by adjusting or closing a switch on the electrosurgical generator utilizing a potentiometer circuit. Still other electrosurgical pencils offer a third button which provides a so-called “blend” waveform or algorithm generally between the cutting waveform and the coagulation waveform.

### SUMMARY

An electrosurgical pencil provided in accordance with aspects of the present disclosure includes a body including proximal and distal ends, the distal end including a pair of supporting arms defining a slot therebetween. A treatment blade extends from a distal end of the body and electrically connects to a first potential of a source of electrosurgical energy, the treatment blade configured to treat tissue upon activation thereof. A protective guard is operably engaged within the slot to the distal end of the body via a pivot pin. The protective guard includes a pair of spaced-apart arms defining a slot therebetween configured to receive the treatment blade. The protective guard is pivotable about the pivot pin between a closed position in which the protective guard encapsulates the treatment blade and at least one second position in which the treatment blade is exposed for tissue treatment.

One or more return electrodes operably connects to the protective guard and is configured to electrically connect to a second potential of the electrosurgical energy source. A biasing member operably couples to the protective guard and is configured to bias the protective guard in the closed position.

In aspects according to the present disclosure, the pair of spaced-apart arms of the protective guard are curved to facilitate pivotable movement thereof when the protective guard is forced against tissue. In other aspects according to the present disclosure, the one or more return electrodes is disposed on an opposite side of the slot defined between the pair of spaced-apart arms of the protective guard. In yet other aspects according to the present disclosure the one or more return electrodes is recessed relative to a leading edge of a corresponding one of the pair of spaced-apart arms of the protective guard. In still other aspects according to the present disclosure, each one of the pair of spaced-apart arms includes a return electrode.

In aspects according to the present disclosure, the treatment blade includes an insulator disposed at a proximal end thereof. In other aspects according to the present disclosure, the treatment blade includes a mechanical profile to facilitate cutting. In yet other aspects according to the present disclosure, the treatment blade is electrically connected to one or more switches disposed on the body that is activatable to supply electrosurgical energy to the treatment member using an energy algorithm. In still other aspects according to the present disclosure, the energy algorithm includes a cutting algorithm, coagulating algorithm or blending algorithm.

In aspects according to the present disclosure, the biasing element is a torsion spring including a first leg operably coupled to the protective guard and a second leg operably coupled to the body.

An electrosurgical pencil provided in accordance with aspects of the present disclosure includes a body including proximal and distal ends. A treatment blade extends from the distal end of the body and electrically connects to a first potential of a source of electrosurgical energy, the treatment blade configured to treat tissue upon activation thereof. A protective guard is operably engaged to the distal end of the body via a pivot pin and includes a pair of spaced-apart arms defining a slot therebetween configured to receive the treatment blade. Each of the spaced-apart arms includes a leading edge and the protective guard is pivotable about the pivot pin between a closed position in which the protective guard encapsulates the treatment blade and a second position in which the treatment blade is exposed for tissue treatment.

One or more return electrodes operably connects to the protective guard and is recessed relative to a leading edge of a respective spaced-apart arm. The one or more return electrodes is configured to electrically connect to a second potential of the electrosurgical energy source. A biasing member is operably coupled to the protective guard and is configured to bias the protective guard in the closed position.

In aspects according to the present disclosure, the pair of spaced-apart arms of the protective guard are curved to facilitate pivotable movement thereof when the protective guard is forced against tissue. In other aspects according to the present disclosure, the one or more return electrodes is disposed on an opposite side of the slot defined between the pair of spaced-apart arms of the protective guard. In still other aspects according to the present disclosure, each one of the pair of spaced-apart arms includes a return electrode.

In aspects according to the present disclosure, the treatment blade includes an insulator disposed at a proximal end thereof. In other aspects according to the present disclosure, the treatment blade includes a mechanical profile to facilitate cutting. In yet other aspects according to the present disclosure, the treatment blade is electrically connected to one or more switches disposed on the body that is activatable to supply electrosurgical energy to the treatment member using an energy algorithm. In still other aspects according to the present disclosure, the energy algorithm includes a cutting algorithm, coagulating algorithm or blending algorithm.

In aspects according to the present disclosure, the biasing member is a torsion spring including a first leg operably coupled to the protective guard and a second leg operably coupled to the body.

### BRIEF DESCRIPTION OF THE DRAWINGS

The accompanying drawings, which are incorporated in and constitute a part of this specification, illustrate embodiments of the present disclosure and, together with the detailed description below, serve to further explain the present disclosure, in which:

FIG. 1 is a perspective view of an electrosurgical pencil including a protective guard according to an exemplary embodiment of the present disclosure;

FIG. 2 is a perspective view of the electrosurgical pencil of FIG. 1 with the protective guard poised for assembly with a pivot pin;

FIG. 3 is a perspective view of the electrosurgical pencil of FIG. 1 shown with the protective guard in a partially open position;

FIG. 4A is a side view of the electrosurgical pencil of FIG. 1 with the protective guard shown in a closed position;

FIG. 4B is a side view of the electrosurgical pencil of FIG. 1 with the protective guard shown in a partially open position;

FIG. 5A is an enlarged, perspective view illustrating a return spring configured to bias the protective guard in a closed position; and

FIG. 5B is an enlarged view of the return spring of FIG. 5A.

### DETAILED DESCRIPTION

As used herein, the term “distal” refers to the portion that is being described which is further from a user, while the term “proximal” refers to the portion that is being described which is closer to a user. Further, to the extent consistent, any of the aspects and features detailed herein may be used in conjunction with any or all of the other aspects and features detailed herein.

As used herein, the terms parallel and perpendicular are understood to include relative configurations that are substantially parallel and substantially perpendicular up to about + or −10 degrees from true parallel and true perpendicular.

“About” or “approximately” as used herein may be inclusive of the stated value and means within an acceptable range of variation for the particular value as determined by one of ordinary skill in the art, considering the measurement in question and the error associated with measurement of the particular quantity (e.g., the limitations of the measurement system). For example, “about” may mean within one or more standard variations, or within ±30%, 20%, 10%, 5% of the stated value.

Descriptions of technical features or aspects of an exemplary embodiment of the present disclosure should typically be considered as available and applicable to other similar features or aspects in another exemplary embodiment of the present disclosure. Accordingly, technical features described herein according to one exemplary embodiment of the present disclosure may be applicable to other exemplary embodiments of the present disclosure, and thus duplicative descriptions may be omitted herein.

Exemplary embodiments of the present disclosure will be described more fully below (e.g., with reference to the accompanying drawings). Like reference numerals may refer to like elements throughout the specification and drawings. The terms “electrostatic pencil” and “electrostatic pen” may be used interchangeably herein.

The present disclosure relates to an electrosurgical pencil with a protective guard that also serves as a local return electrode. A ramped (e.g., curved) surface pushes the guard out of the way when pressed against tissue while the bias of the guard holds it against tissue to ensure proper surface area for energy return.

According to an embodiment of the present disclosure, the electrosurgical pencil includes a spring-loaded cap that protects the surgical blade when the electrosurgical pencil is not in use reducing the possibility of undesirable cutting. The spring-loaded cap includes one or more ground returns disposed proximate the peripheral edges of the protective cap eliminating the need for a patient return pad when activated.

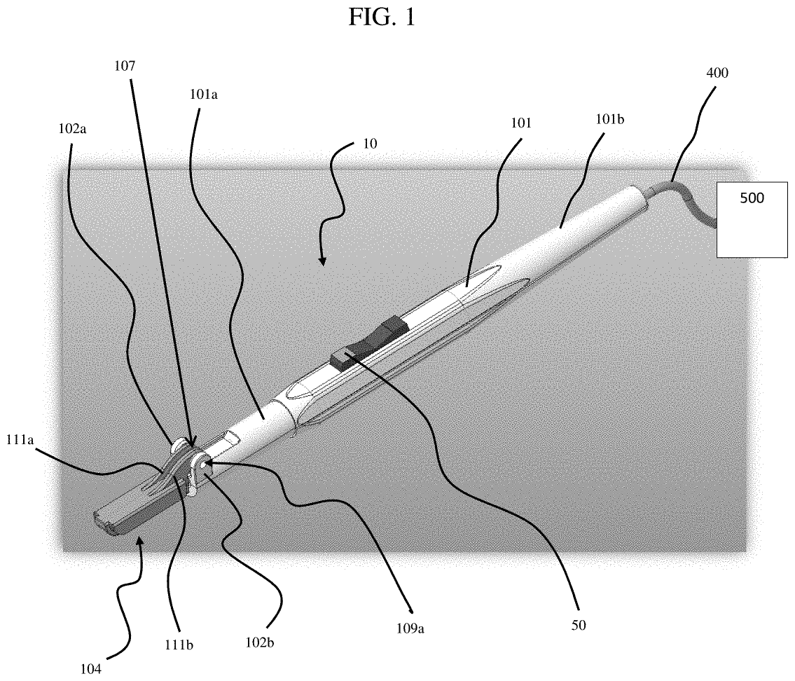

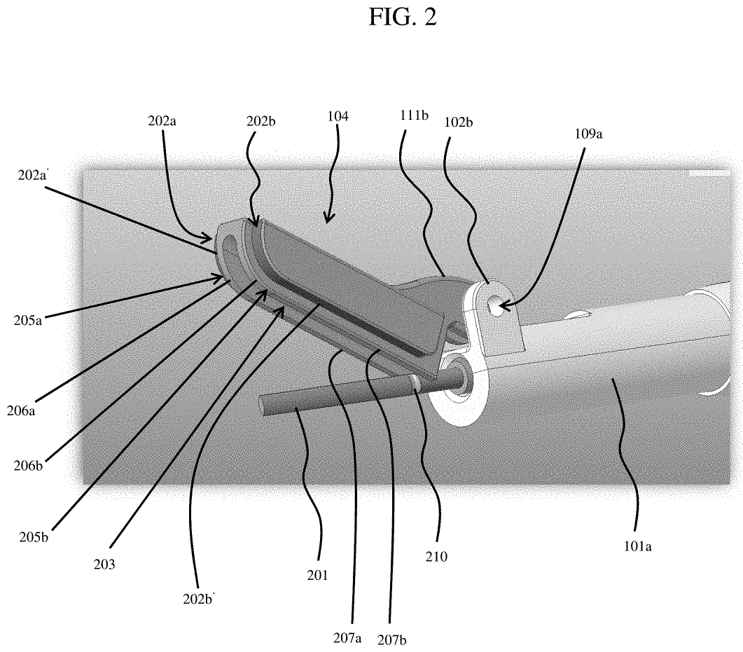

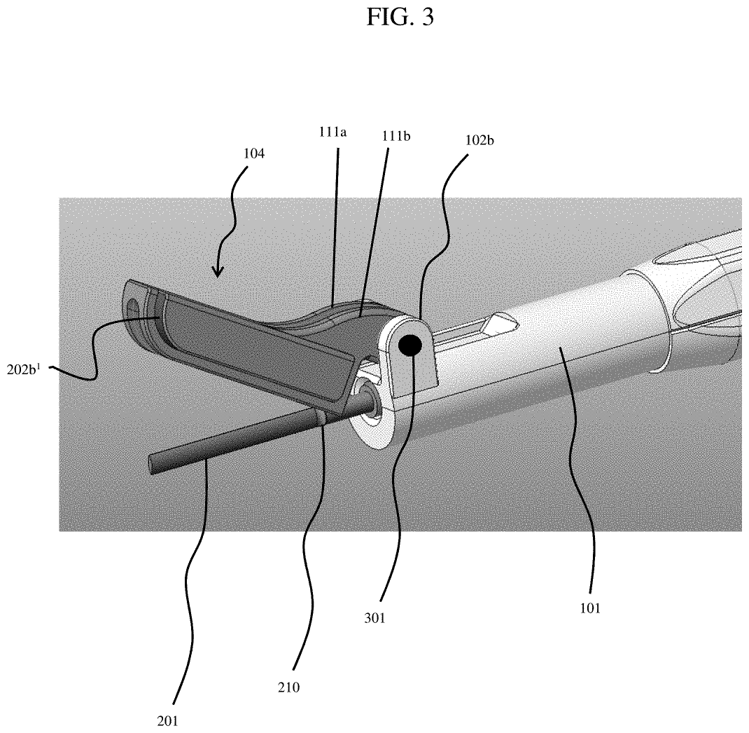

FIG. 1 is a perspective view of an electrosurgical pencil 10 including a protective guard 104 according to an exemplary embodiment of the present disclosure. FIGS. 2-3 are perspective views of the electrosurgical pencil 10 with the protective guard 104 disposed in a partially open position.

Electrosurgical pencil 10 includes a body 101 having distal and proximal ends 101a, 101b, respectively, and a pair of supporting arms 102a and 102b disposed proximate the distal end 101a which define a slot 107 therebetween configured to receive protective guard 104. Supporting arms 102a, 102b each include a pivot hole 109a defined therein configured to receive a corresponding pivot pin 301 for supporting the protective guard 104 in a pivotable fashion.

More particularly, protective guard 104 includes a pair of proximal flanges 111a and 111b that are configured to engage the pair of supporting arms 102a, 102b, respectively, within slot 107. Each flange 111a, 111b includes a corresponding pivot hole (not shown) defined therein that is configured to sit in registry with the pivot holes 109a, 109b of the supporting arms 102a, 102b when the flanges 111a, 111b are seated within slot 107. A pivot pin 301 (See FIG. 3) engages pivot holes 109a, 109b and the pivot holes (not shown) of the flanges 111a, 111b to secure the flanges 111a, 111b to the supporting arm 102a, 102b in pivotable relationship.

Protective guard 104 also includes a pair of spaced apart arms 205a and 205b that extend distally from the proximal flanges 111a, 111b, respectively, and that define a slot 203 therebetween configured to receive and house a treatment member 201, e.g., a cutting blade, as explained in more detail below. The treatment member 201 may be interchangeably referred to as a treatment blade. As defined herein, the treatment member 201 may include a coagulating tip, cutting blade, hook, spatula, etc., depending upon a particular surgical purpose. For the purposes herein, treatment member 201 may described generally as a cutting blade or a treatment blade when describing the figures below.

Each arm 205a, 205b includes a respective distal end 206a and 206b that is generally rounded or otherwise shaped to facilitate rotation of the protective guard 104 when pressure is applied against the distal ends 206a, 206b. Each arm 205a, 205b includes a corresponding return electrode 202a and 202b associated therewith including a respective exposed outer edge 202a′ and 202b′ disposed in opposition to treatment blade 201. During activation and as explained in more detail below, each edge 202a′, 202b′ acts as a potential return path for electrosurgical energy when the treatment blade 201 is activated. The return electrodes 202a, 202b may be recessed relative to a corresponding pair of leading edges 207a and 207b of the protective guard 104 along the lateral surface thereof to reduce the occurrence of an unintentional short.

Treatment blade 201 extends from the distal end 101a of body 101 and is configured to connect to an electrosurgical energy source 500 via one or more leads extending through body 101 and through a cable 400 extending from proximal end 101b of body 101. Treatment blade 201 may be configured to electrically cut tissue but may also include a mechanical edge (e.g., a sharp tip) to facilitate same. Treatment blade 201 includes an insulator 210 disposed at a proximal end thereof configured to insulate the body 101 and other parts of the electrosurgical pencil 10 from stray electrical currents during activation. Insulator 210 may also be configured to releasably couple variously-shaped treatment blade 201 types for different surgical conditions via known mechanical connections, e.g., threadable connection, snap-fit connection, push-lock connection, etc. For example, a cutting blade may be selectively replaced with a more blunt-like blade to simply coagulate tissue if desired.

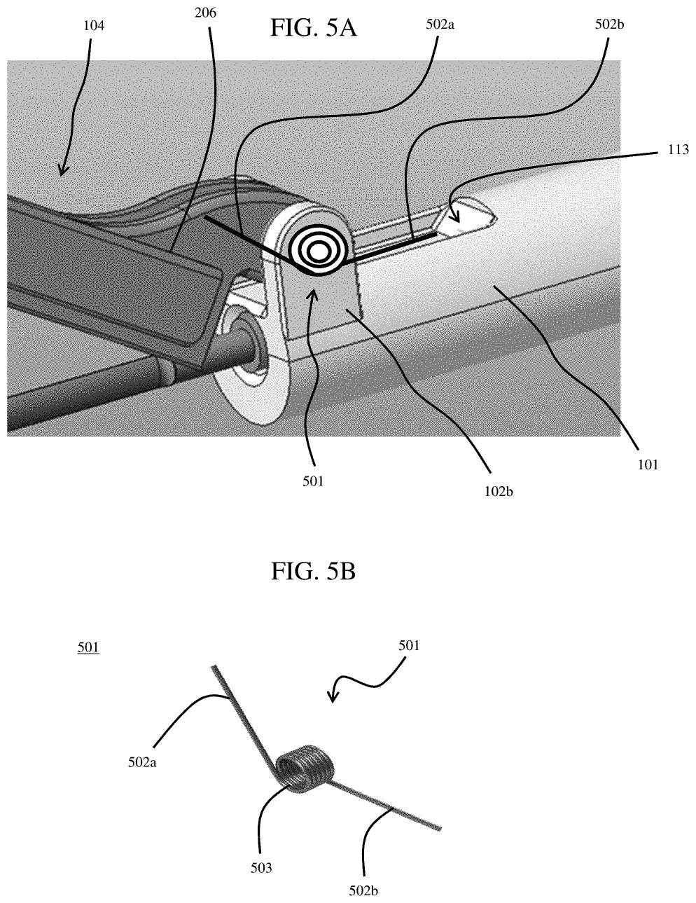

FIG. 3 shows pivot pin 301 engaged within pivot hole 109a and the pivot holes (not shown) of flanges 111a, 111b. As mentioned above, pivot pin 301 enables selective rotation of the protective guard 104 from a safety or closed position to an open position to expose the treatment blade 201 when desired to treat or cut tissue. A return spring 501 (FIGS. 5A and 5B) is utilized to bias the protective guard 104 in a closed position about treatment blade 201. Spring 501 may be one or more torsion springs secured to one or both support arms, e.g., support arm 102b, to bias rotation of the protective guard 104 to the closed position. As shown in FIG. 5B, torsion spring 501 includes a central winding 503 having two legs 502a, 502b that extend in opposite directions therefrom. Leg 502a is configured to engage a trailing edge 206 of return electrode 202b and leg 502b is configured to engage a cavity 113 defined in the distal end 101a of body 101. Torsion springs 501 having various K constants may be utilized depending upon a particular tissue type or desired effect. Other types of springs may also be utilized depending upon a particular purpose or design.

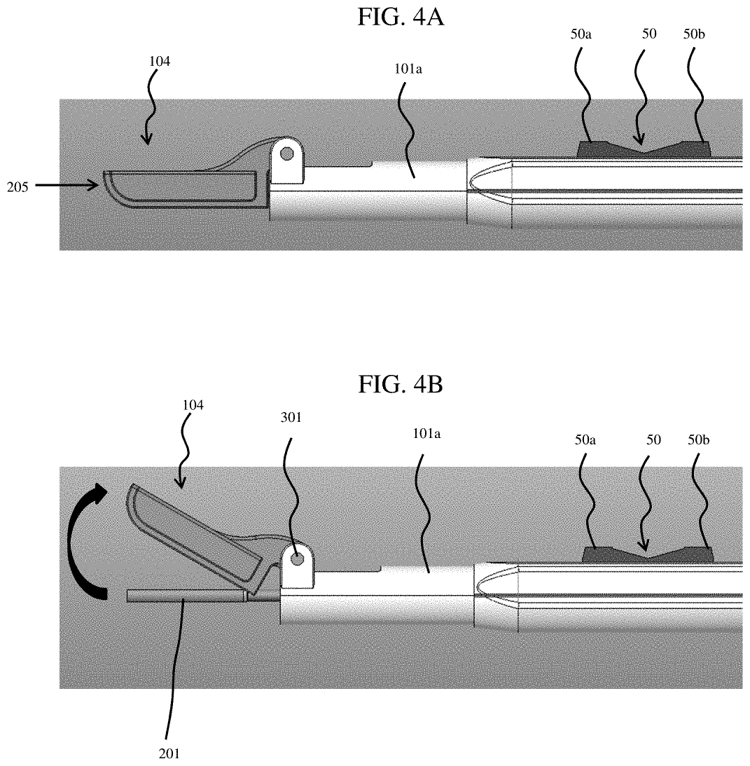

As shown in FIGS. 1, 4A and 4B, electrosurgical pencil 10 also includes a switch, e.g., toggle switch 50, that includes distal and proximal ends 50a and 50b. Any type of switch 50 or switches may be employed depending on a particular surgical purpose of a particular surgical need. As shown, toggle switch 50 is configured to electrically communicate with the electrosurgical energy source 500 (e.g., a generator) to selectively supply energy to the treatment blade 201. When toggled in the distal direction, e.g., toward distal end 50a, an electrical cutting algorithm is employed by the electrosurgical energy source 500. When toggled in the proximal direction, e.g., toward proximal end 50b, an electrical coagulating algorithm is employed by the electrosurgical energy source 500. As can be appreciated, other electrical algorithms may be utilized with other switch types or multiple switch arrangements. For example, U.S. Pat. Nos. 7,244,257, 7,156,842 detail various such switch arrangements for use with electrosurgical pencils, the contents of each of which being incorporated by reference herein.

During use, a surgeon orients the electrosurgical pencil 10 to treat tissue (e.g., coagulate, blend, cut) and pushes the protective guard 104 against the tissue. The rounded configuration of the distal ends 206a, 206b of the protective guard 104 forces the protective guard 104 to rotate proximally against the bias of spring 501 to expose the treatment blade 201, e.g., cutting blade. Energy is then applied by toggling switch 50 in the desired direction to treat tissue. Once tissue treatment is completed, the toggle switch 50 may be activated again to apply a different energy modality or the surgeon can simply disengage the tissue to automatically return (via the bias of spring 501) the protective guard 104 to the closed position about the treatment blade 201.

Thus, the electrosurgical pencil 10 may combine characteristics of both a monopolar and a bipolar electrosurgical pencil and provide predictive treatment, e.g., cutting, without sacrificing cutting efficiency. The exemplary embodiments of the present disclosure are not limited thereto, and the electrosurgical pencil 10 described herein may be efficacious in a wide array of surgical contexts.

The protective guard 104 may be configured as an accessory component configured to be connected to a body of traditional electrosurgical pencil, e.g., added to a pre-existing electrosurgical pencil as an add-on component. Alternatively, the protective guard 104 may be an integrally formed feature of electrosurgical pencil 10.

The various embodiments disclosed herein may also be configured to work with robotic surgical systems and what is commonly referred to as “Telesurgery.” Such systems employ various robotic elements to assist the surgeon and allow remote operation (or partial remote operation) of surgical instrumentation. Various robotic arms, gears, cams, pulleys, electric and mechanical motors, etc. may be employed for this purpose and may be designed with a robotic surgical system to assist the surgeon during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of surgeons or nurses may prep the patient for surgery and configure the robotic surgical system with one or more of the instruments disclosed herein while another surgeon (or group of surgeons) remotely controls the instruments via the robotic surgical system. As can be appreciated, a highly skilled surgeon may perform multiple operations in multiple locations without leaving his/her remote console which can be both economically advantageous and a benefit to the patient or a series of patients.

The robotic arms of the surgical system are typically coupled to a pair of master handles by a controller. The handles can be moved by the surgeon to produce a corresponding movement of the working ends of any type of surgical instrument (e.g., end effectors, graspers, knifes, scissors, etc.) which may complement the use of one or more of the embodiments described herein. The movement of the master handles may be scaled so that the working ends have a corresponding movement that is different, smaller or larger, than the movement performed by the operating hands of the surgeon. The scale factor or gearing ratio may be adjustable so that the operator can control the resolution of the working ends of the surgical instrument(s).

The master handles may include various sensors to provide feedback to the surgeon relating to various tissue parameters or conditions, e.g., tissue resistance due to manipulation, cutting or otherwise treating, pressure by the instrument onto the tissue, tissue temperature, tissue impedance, etc. As can be appreciated, such sensors provide the surgeon with enhanced tactile feedback simulating actual operating conditions. The master handles may also include a variety of different actuators for delicate tissue manipulation or treatment further enhancing the surgeon's ability to mimic actual operating conditions.

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. An electrosurgical pencil, comprising:
a body including proximal and distal ends, the distal end including a pair of supporting arms defining a slot therebetween;
a treatment blade extending from the distal end of the body and electrically connected to a first potential of a source of electrosurgical energy, the treatment blade configured to treat tissue upon activation thereof;
a protective guard operably engaged within the slot to the distal end of the body via a pivot pin, the protective guard including a pair of spaced-apart arms defining a slot therebetween configured to receive the treatment blade, the protective guard pivotable about the pivot pin between a closed position wherein the protective guard encapsulates the treatment blade and at least one second position wherein the treatment blade is exposed for tissue treatment;
at least one return electrode operably connected to the protective guard, the return electrode configured to electrically connect to a second potential of the electrosurgical energy source; and
a biasing member operably coupled to the protective guard and configured to bias the protective guard in the closed position.

2. The electrosurgical pencil of claim 1, wherein the pair of spaced-apart arms of the protective guard are curved to facilitate pivotable movement thereof when the protective guard is forced against tissue.

3. The electrosurgical pencil of claim 1, wherein the at least one return electrode is disposed on an opposite side of the slot defined between the pair of spaced-apart arms of the protective guard.

4. The electrosurgical pencil of claim 1, wherein the at least one return electrode is recessed relative to a leading edge of a corresponding one of the pair of spaced-apart arms of the protective guard.

5. The electrosurgical pencil of claim 1, wherein each one of the pair of spaced-apart arms of the protective guard includes a return electrode.

6. The electrosurgical pencil of claim 1, wherein the treatment blade includes an insulator disposed at a proximal end thereof.

7. The electrosurgical pencil of claim 1, wherein the treatment blade includes a mechanical profile to facilitate cutting.

8. The electrosurgical pencil of claim 1, wherein the treatment blade is electrically connected to at least one switch disposed on the body, the at least one switch activatable to supply electrosurgical energy to the treatment blade using an energy algorithm.

9. The electrosurgical pencil of claim 8, wherein the energy algorithm includes at least one of a cutting algorithm, coagulating algorithm or blending algorithm.

10. The electrosurgical pencil of claim 1, wherein the biasing member is a torsion spring, the torsion spring including a first leg operably coupled to the protective guard and a second leg operably coupled to the body.

11. An electrosurgical pencil, comprising:
a body including proximal and distal ends;
a treatment blade extending from the distal end of the body and electrically connected to a first potential of a source of electrosurgical energy, the treatment blade configured to treat tissue upon activation thereof;
a protective guard operably engaged to the distal end of the body via a pivot pin, the protective guard including a pair of spaced-apart arms defining a slot therebetween configured to receive the treatment blade, each of the spaced-apart arms including a leading edge, the protective guard pivotable about the pivot pin between a closed position wherein the protective guard encapsulates the treatment blade and a second position wherein the treatment blade is exposed for tissue treatment;
at least one return electrode operably connected to the protective guard and recessed relative to a leading edge of a respective spaced-apart arm, the at least one return electrode configured to electrically connect to a second potential of the electrosurgical energy source; and
a biasing member operably coupled to the protective guard and configured to bias the protective guard in the closed position.

12. The electrosurgical pencil of claim 11, wherein the pair of spaced-apart arms of the protective guard are curved to facilitate pivotable movement thereof when the protective guard is forced against tissue.

13. The electrosurgical pencil of claim 11, wherein the at least one return electrode is disposed on an opposite side of the slot defined between the pair of spaced-apart arms of the protective guard.

14. The electrosurgical pencil of claim 11, wherein each one of the pair of spaced-apart arms of the protective guard includes a return electrode.

15. The electrosurgical pencil of claim 11, wherein the treatment blade includes an insulator disposed at a proximal end thereof.

16. The electrosurgical pencil of claim 11, wherein the treatment blade includes a mechanical profile to facilitate cutting.

17. The electrosurgical pencil of claim 11, wherein the treatment blade is electrically connected to at least one switch disposed on the body, the at least one switch activatable to supply electrosurgical energy to the treatment blade using an energy algorithm.

18. The electrosurgical pencil of claim 17, wherein the energy algorithm includes at least one of a cutting algorithm, coagulating algorithm or blending algorithm.

19. The electrosurgical pencil of claim 11, wherein the biasing member is a torsion spring, the torsion spring including a first leg operably coupled to the protective guard and a second leg operably coupled to the body.
